(19) United States
(12) Patent Application Publication
(10) Pub. No.: US 2014/0330098 A1
(43) Pub. Date: Nov. 6, 2014

(54) REFLECTANCE CALIBRATION OF FLUORESCENCE-BASED GLUCOSE MEASUREMENTS

(71) Applicant: CERCACOR LABORATORIES, INC., IRVINE, CA (US)

(72) Inventors: SEAN MERRITT, LAKE FOREST, CA (US); MARCELO LAMEGO, COTO DE CAZA, CA (US)

(21) Appl. No.: 14/330,273

(22) Filed: Jul. 14, 2014

Related U.S. Application Data
(63) Continuation of application No. 12/511,742, filed on Jul. 29, 2009.

(51) Int. Cl. A61B 5/145; A61B 5/00; A61B 5/1455

(52) U.S. Cl. CPC A61B 5/14532; A61B 5/1455; A61B 5/0071
USPC 600/316

(57) ABSTRACT

A noninvasive or minimally invasive procedure and system for measuring blood glucose levels is disclosed. A set of photodiodes detects the fluorescence and reflectance of light energy emitted from one or more emitters, such as LEDs, into a patient's skin. In an embodiment, small molecule metabolite reporters (SMMRs) that bind to glucose are introduced to the measurement area to provide more easily detected fluorescence.

### REFERENCE TO RELATED APPLICATIONS

This application is a continuation of U.S. patent application Ser. No. 12/511,742, filed Jul. 29, 2009, entitled “REFLECTANCE CALIBRATION OF FLUORESCENCE-BASED GLUCOSE MEASUREMENTS,” which is hereby incorporated herein by reference in its entirety.

### BACKGROUND

1. Field

The disclosure relates to measurement of an in vivo glucose level by emitting an excitation wavelength and measuring a fluorescence emission.

2. Description of the Related Art

Identifying and understanding the risk factors associated with diabetes is invaluable for the development and evaluation of effective intervention strategies. Lacking normal regulatory mechanisms, diabetics are encouraged to strive for optimal control through a modulated life style approach that focuses on dietary control, exercise, and glucose self-testing with the timely administration of insulin or oral hypoglycemic medications. Invasive forms of self-testing are painful and fraught with a multitude of psychosocial hurdles, and are resisted by most diabetics. Alternatives to the currently available invasive blood glucose testing are highly desirable.

Conventional approaches seek to reduce or eliminate the skin trauma, pain, and blood waste associated with traditional invasive glucose monitoring technologies. In general, non-invasive optical blood glucose monitoring requires no samples and involves external irradiation with electromagnetic radiation and measurement of the resulting optical flux. Glucose levels are derived from the spectral information following comparison to reference spectra for glucose and background interferants, reference calibrants, and/or application of advanced signal processing mathematical algorithms. Candidate radiation-based technologies include: 1) mid-infrared (MIR) spectroscopy, 2) near-infrared (NIR) spectroscopy, 3) far-infrared (FIR) spectroscopy, 4) radio wave impedance, 5) infrared photoacoustic spectroscopy and 6) Raman spectroscopy. Each of these methods uses optical sensors, and relies on the premise that the absorption pattern of infrared light (700-3000 nm) can be quantitatively related to the glucose concentration. Other substances, such as water, protein, and hemoglobin, are known to absorb infrared light at these wavelengths and easily obscure the relatively weak glucose signal.

Other approaches are based on microvascular changes in the retina, acoustical impedance, NMR spectroscopy, and optical hydrogels that quantify glucose levels in tear fluid. While putatively non-invasive, these technologies have yet to be demonstrated as viable in clinical testing.

Nearly non-invasive techniques tend to rely on interstitial fluid extraction from skin. This can be accomplished using permeability enhancers, sweat inducers, and/or suction devices with or without the application of electrical current. One device recently approved by the FDA relies on reverse iontophoresis, utilizing an electrical current applied to the skin. The current pulls out salt, which carries water, which in turn carries glucose. The glucose concentration of this extracted fluid is measured and is proportional to that of blood. This technology, in keeping with its nearly non-invasive description, is commonly associated with some discomfort and requires at least twice daily calibrations against conventional blood glucose measurements (e.g., invasive lancing).

Other nearly non-invasive blood glucose monitoring techniques similarly involve transcutaneous harvesting for interstitial fluid measurement. Other technologies for disrupting the skin barrier to obtain interstitial fluid include: 1) dissolution with chemicals; 2) microporation with a laser, sound, or electrical stimulation; 3) penetration with a thin needle; and/or 4) suction with a pump. Minimally invasive blood glucose monitoring can also involve the insertion of an indwelling glucose monitor under the skin to measure the interstitial fluid glucose concentration. These monitors typically rely on optical or enzymatic sensors. Technologically innovative, these in situ sensors have had limited success. Implantable glucose oxidase sensors have been limited by local factors causing unstable signal output, whereas optical sensors must overcome signal obfuscation by blood constituents as well as interference by substances with absorption spectra similar to glucose. Moreover, inflammation associated with subcutaneous monitoring may contribute to systematic errors requiring repositioning, recalibration or replacement, and more research is needed to evaluate the effects of variable local inflammation at the sensor implantation site on glucose concentration and transit time.

Interstitial fluid glucose concentrations have previously been shown to be similar to simultaneously measured fixed or fluctuating blood glucose concentrations (Bantle et al., Journal of Laboratory and Clinical Medicine 130:436-441, 1997; Sternberg et al., Diabetes Care 18:1266-1269, 1995). Such studies helped validate non-invasive/minimally invasive technologies for blood glucose monitoring, insofar as many of these technologies measure glucose in blood as well as interstitial fluid.

A non-invasive glucose monitor that is portable, simple and rapid to use, and that provides accurate clinical information is highly desirable. In particular, the ability to derive primary and secondary order information regarding real time, dynamic glucose metabolism (such as the direction and rate of change of bioavailable glucose distributed within the blood and interstitial fluid space) is highly desirable.

### SUMMARY

A noninvasive or minimally invasive procedure and system for measuring blood glucose levels is disclosed. A set of photodiodes detects the fluorescence and reflectance of light energy emitted from one or more emitters, such as LEDs, into a patient's skin. In an embodiment, small molecule metabolite reporters (SMMRs) that bind to glucose are introduced to the measurement area to provide more easily detected fluorescence.

### BRIEF DESCRIPTION OF THE DRAWINGS

FIG. 1 depicts a block diagram of a method of measuring a glucose level with fluorescence and reflectance measurements.

FIG. 2 depicts an embodiment of a system for in vivo measurement of a glucose level with fluorescence and reflectance measurements.

FIG. 3 depicts a set of test glucose measurement results that have not been normalized with a reflectance measurement.

FIG. 4 depicts a set of test glucose measurement results normalized with a reflectance measurement.

FIG. 5 depicts a block diagram of a method of measuring a glucose level with first and second fluorescence and reflectance measurements.

FIG. 6 depicts a system for in vivo measurement of a glucose level with first and second fluorescence and reflectance measurements.

FIG. 7 depicts a method of normalizing a fluorescence measurement with a reflectance measurement and background fluorescence and reflectance measurements.

FIG. 8 depicts an apparatus for measuring a glucose level.

### DETAILED DESCRIPTION

Tissue fluorescence measurements are calibrated to account for instrument effects, which may include differences in source intensity, detector gain, molecule concentration, or measurement device location relative to the fluorescing molecule on the skin.

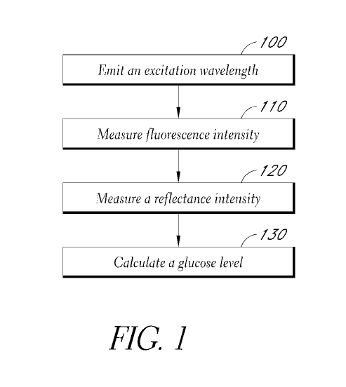

FIG. 1 depicts a method of measuring a glucose level. An excitation wavelength is emitted 100 to stimulate fluorescence and reflectance responses. A fluorescence intensity is measured 110. A reflectance intensity is measured 120. To obtain the most valuable results, the reflectance intensity measurement 120 and fluorescence intensity measurement 110 probe essentially the same volume or surface area. The tissue reflection measurement 120 varies with the instrument response of the system, as well as the molecule concentration and the location of the measurement device, in a manner that is directly related to the measured fluorescence intensity resulting from measurement 110 of the molecule. A first approximation of the relationship between the fluorescence intensity and the reflectance intensity is linear.

A glucose level is calculated 130 with the reflectance intensity information and fluorescence intensity information. In an embodiment, the ratio of fluorescence intensity to reflectance intensity is used to help filter out background readings. This is often plotted against sample glucose measurements from direct blood testing of a number of test subjects. With a large enough sample size, a best fit line or curve can be determined to plot the fluorescence intensity/reflectance intensity ratio against glucose levels. This data can then in turn be used to calculate glucose levels based on the noninvasive fluorescence and reflectance intensity readings; the data is generally known as a calibration curve. By taking the ratio of the fluorescence measurement (emission wavelengths) with the reflectance measurement at the excitation wavelength, the measurement is calibrated and measurement error reduced.

In one embodiment, the same excitation source is used to stimulate both the absorption and fluorescence measurements, but different detectors are used to filter wavelength intensities at different points in the spectrum, corresponding to the fluorescence and reflectance emissions of the targeted tissue. For reflectance intensity measurements, the detector will typically measure the intensity of a wavelength at approximately the same wavelength as the excitation source. For fluorescence measurements, the measured wavelength or wavelengths preferably corresponds to those wavelengths at which the fluorescing compound most accurately reflects a glucose level. Indeed, for the most accurate measurements, it is advantageous to use an excitation source at more than one wavelength or a spectrum of wavelengths, and a measurement device capable of measuring reflectance and fluorescence intensity at a spectrum of wavelengths.

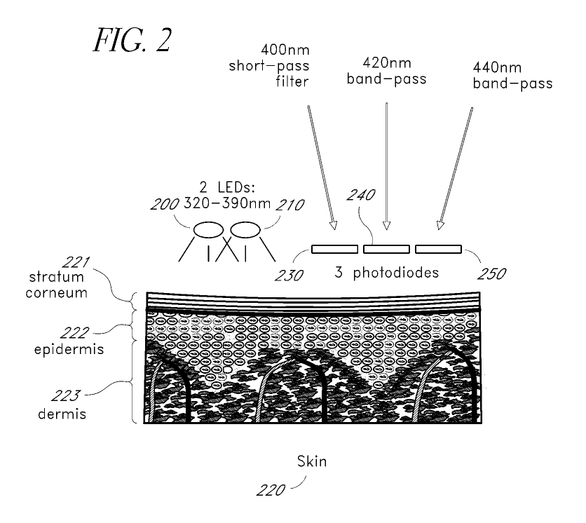

FIG. 2 depicts a general overview of an embodiment of a device for noninvasive or minimally invasive in vivo measurement of a glucose level. A first LED 200 and a second LED 210 emit excitation wavelengths, preferably, in an embodiment, between about 320 and about 390 nanometers. The excitation wavelengths from first LED 200 and second LED 210 are directed at skin 220. Skin 220 is generally comprised of the 5 outermost flat areas of the stratum corneum 221, the epidermis 222 below that, and the dermis 223 below that. Each of these layers will reflect some of the light emitted from the first and second LEDs 200, 210 due to the scattering of the tissue. As such, in an embodiment, the system has a short-pass filter 230 to measure reflected wavelengths below 400 nm. In an embodiment short-pass filter 230 is a photodiode.

In addition, compounds in skin 220 fluoresce as a result of their interaction with the excitation wavelength. Some of these fluorescing compounds emit a fluorescence signal corresponding to an in vivo glucose level. As such, the system of FIG. 2 also includes two band-pass filters 240, 250 to detect the fluorescence at various wavelengths. In an embodiment, these filters 240, 250 are photodiodes.

It is also possible to introduce compounds into the skin called Small Molecule Metabolite Reporters (SMMRs) that bind with glucose and yield a more distinct fluorescence spectrum than compounds existing naturally in skin 220. In an embodiment, SMMRs are delivered to the tissue of the stratum corneum 221 and the epidermis 222. Therefore it is preferable to configure the LEDs 200, 210 and photodiodes 230, 240, 250 to most effectively probe the stratum corneum 221 and epidermis 222. A separation between the LEDs 200, 210 and filters 230, 240, 250 can help determine the penetration depth of the light field in the tissue.

At the energy level that it absorbs, the SMMR is a high absorber of energy. Thus, the greater the concentration of SMMR, as it is bound to glucose, the less the reflectance measurement. An example of such an SMMR is ARG327D. In one embodiment, the SMMR is injected with a micro-needle. In other instances, SMMR is brushed, wiped, or tattooed onto skin 220. In an embodiment of the system of FIG. 2, the SMMR fluoresces in reaction to an excitation wavelength of approximately 350 nm. The SMMR yields valuable fluorescence intensity data at approximately 420 nm and 440 nm. Thus, in the embodiment, the device has a first band-pass filter 240 at about 420 nm and a second band-pass filter 250 at about 440 nm. Because the excitation wavelength is at approximately 320-390 nm, neither first band-pass filter 240 nor second band-pass filter 250 will register extraneous reflectance wavelengths.

An embodiment of the system may utilize 2 LEDs to enable the reflectance and fluorescence measurement to probe the same region, such as in a cross pattern. Typically it is preferred that these LEDs 200, 210 would be the same wavelength.—Additional LEDs are more likely to be redundant rather than provide significant additional information, so embodiments with three or more LEDs are less preferred. A full spectrum of photodiodes is very desirable, however. Broadband spectra for detection are possible by using a spectrometer for detection. A monochromator is an example of a light energy emitter that can take the place of one or more LEDs 200, 210.

In addition, the wavelengths measured by the various filters vary with the spectra emitted by the fluorescing molecules. As described earlier, with reference to FIG. 1, any number of photodiodes may be used, depending on the desired resolution and accuracy of the measured spectrum. So, another example of the system of FIG. 2 uses a fluorimeter with multispectral filters capable of reading an entire fluorescence spectrum. This embodiment is advantageous in hospitals or other settings where the accuracy and precision of glucose measurements are imperative and the expense of the instrument can be defrayed by use with a large number of patients. Calibration equations for a multispectral embodiment correspond to those for a single wavelength application and are discussed in greater detail below. The multispectral data fitting would be comparable to using only one ore two photodiodes.

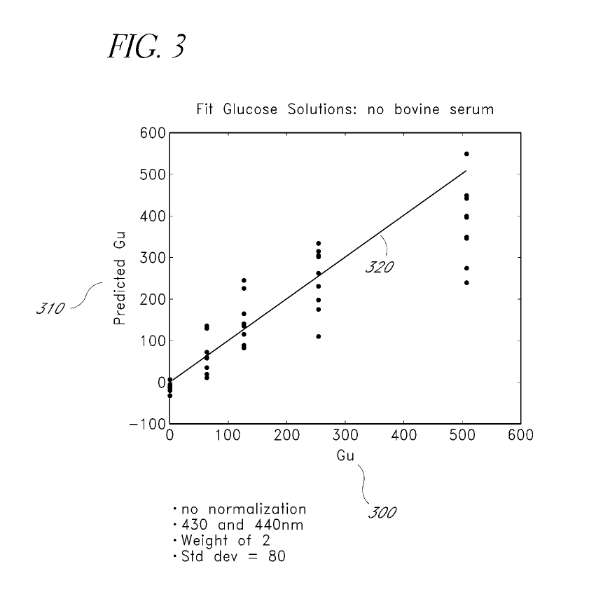

FIG. 3 is a chart of glucose measurements made based on fluorescence intensity, without reflectance calibration. The measurements were made on test samples and are correlated to direct measurements as described above. The excitation wavelength is 350 nm, and the fluorescence intensity is measured at 430 and 440 nm. The values on the X-axis are actual glucose levels Gu 300. The values on the Y-axis are predicted glucose values Gu 310. Of course, the predicted glucose values 310 are the same as the actual glucose values 300. This linear relationship is depicted as line 320. Clusters of measured glucose levels, based on the measured fluorescence intensity of SMMR compounds, are represented by dots on the chart. As can be seen, glucose measurements are given for actual glucose levels 300 of approximately 75, 125, 250, and 500 mg/dL. At glucose level zero, the measured glucose values are relatively tightly packed around the predicted glucose level of zero. However, as the actual glucose level rises, the accuracy of the measured glucose levels decreases. At the highest glucose level of 500, the precision of measured results also decreases, as almost all data points are below the predicted glucose level of 500.

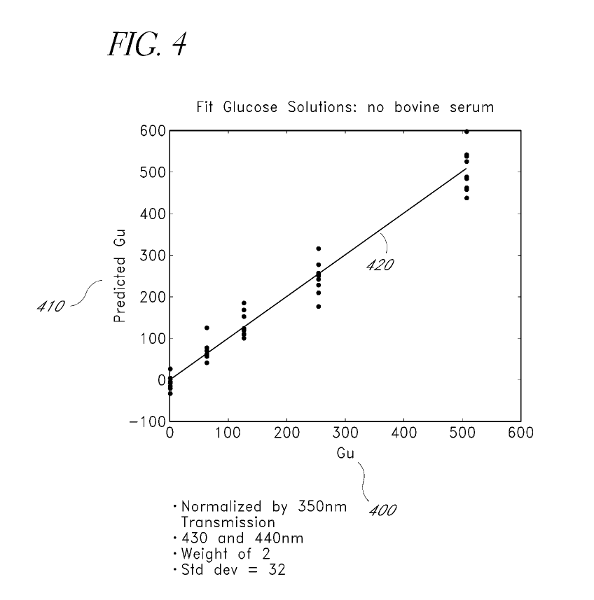

FIG. 4 is another chart of glucose measurements based on fluorescence intensity. However, the test results in FIG. 4 are calibrated with a reflectance intensity measurement taken at a wavelength of 350 nm—that is, at approximately the excitation wavelength. Once again, fluorescence intensity is measured at 430 and 440 nanometers. The X-axis is an actual glucose level 400, and the Y-axis is a predicted glucose level 410. The linear, equal relationship between the actual glucose level 400 and predicted glucose level 410 is indicated by line 420. The primary feature of the test results in FIG. 4 is the much-improved accuracy and precision of the measured glucose levels when the fluorescence intensity is calibrated with a reflectance intensity.

As with the results depicted in FIG. 3, the measured glucose levels correspond quite closely to an actual glucose level of zero. However, for higher glucose levels—like 75, 125, 250, and 500—the measured readings are clustered much more closely around the predicted glucose level 420. Indeed, at actual glucose levels of 75, 125, and 250 the calibrated glucose measurements are mostly tightly bunched around the predicted glucose level 420. The precision of the measurements at the highest actual glucose level depicted is also improved, as half of the measurements are above the predicted glucose level and half below.

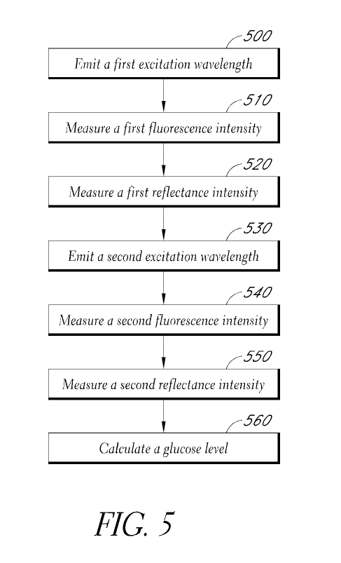

FIG. 5 is another method of measuring in vivo glucose level. Like the method depicted in FIG. 1, it includes measuring both a fluorescence and reflectance intensity. In addition, the method includes measuring a second fluorescence and reflectance intensity to normalize data from the first set of measurements. For example, first fluorescence and reflectance intensity measurements are taken at a site treated with an SMMR. Second fluorescence and reflectance intensity measurements are taken at an untreated, background site to determine the natural fluorescence and reflectance properties of the skin.

Skin naturally has a background tissue fluorescence and absorption that originates from different tissue fluorophores such as collagen, FAD, and NADH, and absorbers such as hemoglobin. These fluorophores and absorbers all have different emission and absorption profiles that are distinct with wavelength. Different concentrations of background fluorophores and absorbers in different skin types may interfere with the fluorescence and reflectance signals that are being measured from a glucose-binding fluorophore in the skin. In order to correct for background fluorescence and reflectance, separate fluorescence and reflectance measurements are made at a tissue site that has no glucose-binding molecule. The background measurement is then used to correct for the background tissue fluorescence and absorption through a wavelength normalization.

In the method of FIG. 5, a first excitation wavelength is emitted 500. A first fluorescence intensity is measured 510. A first reflectance intensity is measured 520. Then, a second excitation wavelength is emitted 530, a second fluorescence intensity is measured 540, and a second reflectance intensity is measured 550. From the various fluorescence and reflectance measurements, a glucose level is calculated 560.

Persons of skill will appreciate that no particular ordering is necessarily implied in the operations depicted in either FIG. 5 or the earlier described FIG. 2. In another example, the background fluorescence and intensity measurements are made before fluorescence and intensity measurements at the SMMR-treated site. Or, the reflectance intensity is measured before or concurrently with the fluorescence intensity. In other embodiments, the glucose-calculations are segmented into various points within the method.

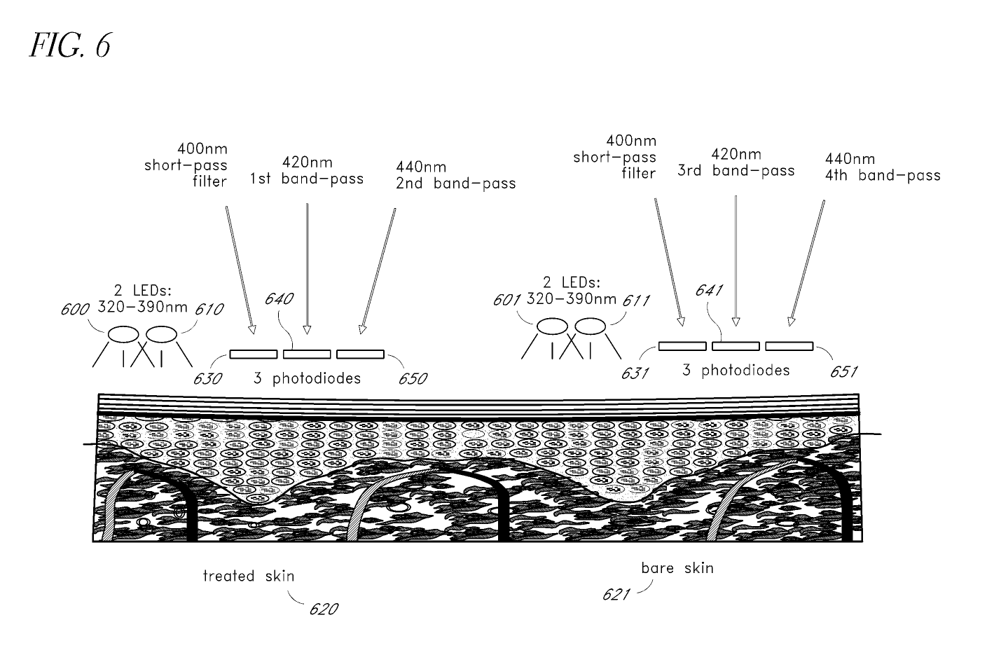

FIG. 6 depicts a general overview of another embodiment of a device for noninvasive or minimally invasive measurement of a glucose level. The system illustrated measures an in vivo glucose level using fluorescence and reflectance measurements at both a treated and untreated skin site. In this embodiment, a first LED 600 and second LED 610 emit excitation signals between 320 nm and 390 nm. First LED 600 and second LED 610 are directed at an area of treated skin 620. Treated skin 620 is treated with a glucose-binding fluorophore, like an SMMR. When it absorbs the excitation signal, the glucose-binding fluorophore emits a fluorescence spectrum. A first band-pass filter 640 at 420 nm and a second band-pass filter 650 at 440 nm measure the intensity level at two points along the fluorescence spectrum. In general, the intensity levels correspond with a glucose level. Treated skin 620 also reflects some of the excitation wavelengths at between 320 nm to 390 nm emitted by first LED 600 and second LED 610. Short-pass filter 630 measures reflectance intensity at wavelengths shorter than 400 nm.

In addition to the fluorescence and reflectance measurements made at treated skin site 620, measurements are made at a bare skin site 621. A third LED 601 and fourth LED 611 generate excitation wavelengths at between 320 nm and 390 nm. Typically, the excitation wavelength of first LED 600 is the same as the excitation wavelength of third LED 601, and the excitation wavelength of second LED 610 is the same as the excitation wavelength of fourth LED 611. In one embodiment, the same excitation apparatus is used to measure different skin sites at different times. In this embodiment, first LED 600 is the same as third LED 601, and second LED 610 is the same as fourth LED 611.

Third LED 601 and fourth LED 611 excite fluorophores like collagen and others mentioned earlier within bare skin 621. The fluorophores emit fluorescent spectra. A third band-pass filter 641 and fourth band-pass filter 651 measure the emitted fluorescent spectra at 420 nm and 440 nm, respectively.

Bare skin 621 reflects some of the excitation wavelengths emitted by third LED 601 and fourth LED 611. A second short-pass filter 631 measures reflectance intensity at wavelengths shorter than 400 nm.

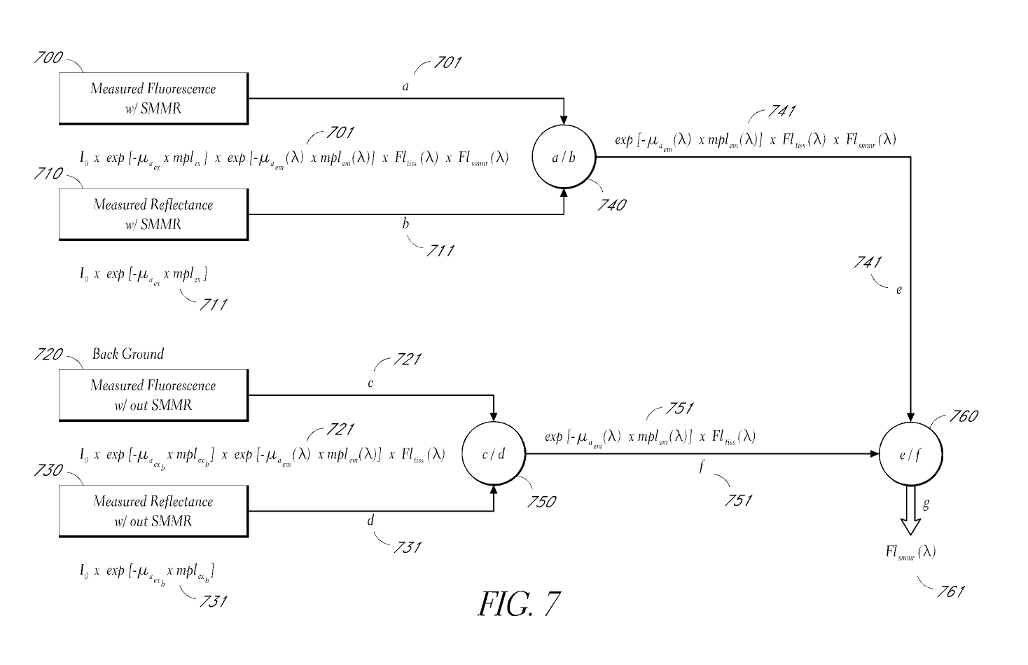

FIG. 7 depicts a glucose level calculation using first and second reflectance and absorption intensity measurements. The equation of FIG. 7 begins with four familiar components: (1) a first measured fluorescence 700, at a treated skin site; (2) a first measured reflectance 710, at a treated skin site; (3) a second, background fluorescence measurement 720, at a bare skin site; and (4) a second, background reflectance measurement 730, at a bare skin site.

Equation a 701 is the measured fluorescence at a tissue site that contains SMMRs. Variable I₀ is excitation beam intensity. Variable μₐ_{ₑₓ} is the absorption coefficient of tissue and SMMR at excitation wavelengths. Variable mplₑₓ is the mean path length of light at excitation wavelength in tissue containing SMMR. Variable λ is the emission wavelength. Variable μₐ_{ₑₘ}(λ) is the absorption coefficient of tissue at emission wave lengths. Variable mplₑₘ(λ) is the mean path length of light at emission wavelengths. Variable Flₜᵢₛₛ(λ) is the tissue fluorescence intensity at emission wavelength. And, variable Flₛₘₘᵣ(λ) is the smmr fluorescence intensity at emission wavelength.

The other equations depicted in FIG. 7 use comparable variables to Equation a 701. In addition, Equation c 721 and Equation d 731 use variable

$${μ}_{{a}_{{ex}_{b}}}$$

for the absorption coefficient of tissue at excitation wavelength without an SMMR, and variable mplₑₓ_{_{b}} for the mean path length of light at excitation wavelength in tissue without an SMMR.

Because the measured reflectance 710 with SMMR and measured reflectance 730 without SMMR do not attempt to measure a fluorescence spectra, Equation b 711 and Equation d 731 that correspond to those measurements are not factors of variables that depend on an emission wavelength λ. Instead, both reflectance measurements are the product of the excitation beam intensity I₀ and the exponential function of the product of the tissue's absorption coefficient λₐ_{ₑₓ} and the mean path length of light in tissue mplₑₓ at the excitation wavelength.

The measured fluorescence 700 with SMMR and the measured reflectance 710 with SMMR are normalized 740 through a ratio of Equation a 701 over Equation b 711. The normalization 740 results in Equation e 741. Equation e 741 removes dependence of effective light source intensity that includes absorption effects of SMMR and tissue at the excitation wavelength.

Similarly, background measure fluorescence 720 without SMMR and background measured reflectance 730 without SMMR are normalized 750 through a ratio of Equation c 721 over Equation d 731. Normalization 750 results in Equation f 751. Equation f 751 removes dependence of effective light source intensity that includes absorption effects of tissue at excitation wave length.

Equation e 741 and Equation f 751 are normalized 760 through a ratio of Equation e 741 over Equation f 751. Normalization 760 results in Equation g 761. Equation g 761 is the SMMR fluorescence intensity at an emission wave length, calibrated with a reflectance measurement and corrected with a measurement at a bare-skin, background site. As explained above, in some embodiments, equation g is correlated to a glucose level of the blood through the use of a calibration curve determined from the empirical glucose measurements gathered from direct blood testing and compared to the less invasive or noninvasive measurements. The glucose value can then be output to a user, such as to allow monitoring of the patient.

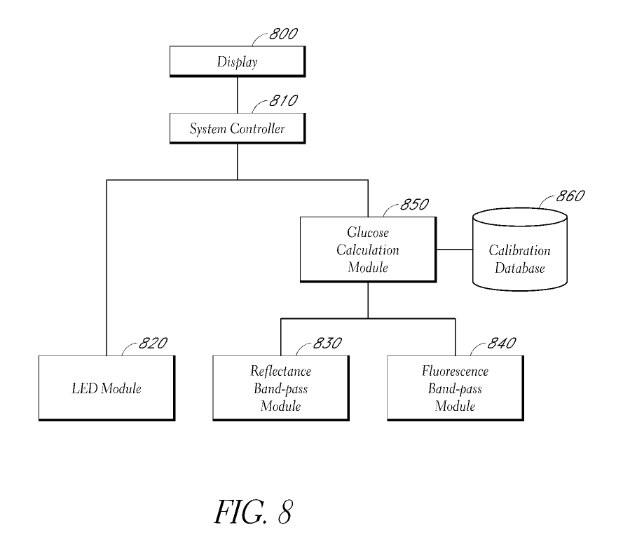

FIG. 8 is an apparatus for measurement of an in vivo glucose level. Among other functions, display 800 shows a glucose level based on a fluorescence intensity measurement. System controller 810 connects to display 800 and the various other modules that measure a glucose level. System controller 810 connects to an LED module 820, which emits one or more excitation wavelengths. Examples of components comprising an LED module are the first LED 200 and second LED 210 in FIG. 2.

The device in FIG. 8 also has a glucose calculation module 850. Glucose calculation module 850 connects to a reflectance band-pass module 830 and fluorescence band-pass module 840. Reflectance band pass module 830 measures a reflectance wavelength intensity. Fluorescence band-pass module 840 measures a fluorescence emission intensity. An example of a component comprising a reflectance band-pass module 830 is short-pass filter 230 from FIG. 2. Examples of components comprising a fluorescent band-pass module 840 include first band-pass filter 240 and second band pass filter 250 from FIG. 2.

Reflectance band-pass module 830 and fluorescence band-pass module 840 relay measured wavelength intensity data to glucose calculation module 850. Glucose calculation module 850 uses these measurements, along with excitation data from LED module 820, to calculate a glucose level. In doing so, glucose calculation module 850 accesses a calibration database 860. Calibration database 860 includes, for instance, data from previous measurements or samples from other subjects or population groups that are used to further calibrate a glucose-level measurement. The glucose calculation module 850 relays glucose-level data back to system controller 810 for presentation on display 800.

Although a glucose monitor and method have been disclosed in detail in connection with various embodiments of the present disclosure, one of ordinary skill in the art will appreciate many variations and modifications within the scope of this disclosure. These embodiments are disclosed by way of example only and do not limit the scope of the disclosure, which is defined by the claims that follow.

What is claimed is:

1. A method of measuring a glucose level, comprising:
directing a first excitation wavelength at a first skin location within a stratum corneum skin layer;
measuring a first fluorescence intensity from the first skin location;
measuring a first reflectance intensity from the first skin location; and
calibrating the first fluorescence intensity using the first reflectance intensity to determine a first glucose level.

2. The method of claim 1, further comprising delivering to the first skin location a quantity of small molecule metabolite reporters (SMMRs) configured to bind to glucose.

3. The method of claim 2, wherein delivering to the first skin location the quantity of SMMRs comprises at least one of brushing and wiping the SMMRs to the first skin location.

4. The method of claim 1, wherein calibrating the first fluorescence intensity comprises calculating a first ratio, wherein a numerator of the first ratio is the first fluorescence intensity and a denominator of the first ratio is the first reflectance intensity.

5. The method of claim 1, further comprising:
directing a second excitation wavelength at a second skin location within the stratum corneum skin layer;
measuring a second fluorescence intensity from the second skin location; and
measuring a second reflectance intensity from the second skin location.

6. The method of claim 5, wherein no small molecule metabolite reporters (SMMRs) are delivered the second skin location.

7. The method of claim 5, further comprising calibrating the first glucose level with the second fluorescence intensity and the second reflectance intensity.

8. The method of claim 7, wherein calibrating the first glucose level comprises:
calculating a second ratio comprising the second fluorescence intensity and the second reflectance intensity; and
calculating a third ratio comprising the first ratio as a numerator of the third ratio and the second ratio as a denominator of the third ratio.

9. The method of claim 1, wherein directing the first excitation wavelength at the first skin location comprises directing a wavelength from 320 nanometers (nm) to 390 nm.

10. A system for measuring a glucose level, comprising:
an excitation module configured to direct a first excitation signal at a first skin location within a stratum corneum skin layer for probing a fluorophore at the first skin location;
a fluorescence measurement module configured to measure a first fluorescence intensity emitted from the first skin location;
a reflectance measurement module configured to measure a first reflectance intensity emitted from the first skin location; and
a glucose calculation module configured to determine a first measured glucose level using the first fluorescence intensity and the first reflectance intensity.

11. The system of claim 10, wherein the first skin location comprises small molecule metabolite reporters (SMMRs), and wherein the first excitation signal is configured to probe the SMMRs.

12. The system of claim 10, wherein the excitation module is configured to emit the first excitation signal at a wavelength of 320 nanometers (nm) to 390 nm.

13. The system of claim 12, wherein the excitation module further comprises two light emitting diodes (LEDs) each configured to emit the first excitation signal at the wavelength of 320 nanometers (nm) to 390 nm.

14. The system of claim 10, wherein the glucose calculation module is further configured to calibrate the first fluorescence intensity using the first reflectance intensity by calculating a first ratio, wherein a numerator of the first ratio is the first fluorescence intensity and a denominator of the first ratio is the first reflectance intensity.

15. The system of claim 10, wherein the excitation module is further configured to direct a second excitation signal at a second skin location within the stratum corneum skin layer, wherein the fluorescence measurement module is further configured to measure a second fluorescence intensity from the second skin location; and wherein the reflectance measurement module is further configured to measure a second reflectance intensity from the second skin location.

16. The system of claim 15, wherein the second excitation signal has a same wavelength as the first excitation signal.

17. The system of claim 15, wherein the glucose calculation module is further configured to calibrate the first measured glucose level using the second fluorescence intensity and the second reflectance intensity by calculating a second ratio using the second fluorescence intensity and the second reflectance intensity.

18. The system of claim 15, wherein the second skin location is a background skin location lacking small molecule metabolite reporters (SMMRs).

19. The system of claim 10, wherein the calculation module is further configured to compare the calibrated first measured glucose level with a calibration curve for determining a glucose reading for the body.

20. The system of claim 10, wherein the fluorescence measurement module is configured to measure emitted wavelengths at 430 nanometers (nm) and 440 nm.
